(19) United States
(12) Patent Application Publication
(10) Pub. No.: US 2025/0012805 A1
(43) Pub. Date: Jan. 9, 2025

(54) PRODUCTS AND USES THEREOF FOR PREDICTING THE SENSITIVITY OF A SUBJECT TO CANCER IMMUNOTHERAPY AND FOR SELECTING OPTIMIZED THERAPY

(71) Applicants: INSTITUT GUSTAVE ROUSSY, VILLEJUIF (FR); INSTITUTE NATIONAL DE LA SANTE ET DE LA RECHERCHE MEDICALE, PARIS (FR); UNIVERSITE PARIS-SACLAY, GIF-SUR-YVETTE (FR)

(72) Inventors: LAMBROS TSELIKAS, CHARENTON LE PONT (FR); FRANÇOIS-XAVIER DANLOS, MONTREUIL (FR); DELPHINE BREDEL, VILLEJUIF (FR); SANDRINE SUSINI, PARIS (FR); MÉLODIE BONVALET, VERRIERES-LE-BUISSON (FR); SÉVERINE MOURAUD, CLAMART (FR); CAROLINE ROBERT, FONTENAY-AUX-ROSES (FR); AURÉLIEN MARABELLE, SCEAUX (FR)

(21) Appl. No.: 18/708,619

(22) PCT Filed: Nov. 10, 2022
(86) PCT No.: PCT/EP2022/081379

§ 371 Date: May 9, 2024

(30) Foreign Application Priority Data
Nov. 10, 2021 (EP) 21306577.4

(51) Int. Cl. G01N 33/574

(52) U.S. Cl. CPC G01N 33/57484; G01N 2333/475; G01N 2333/5412; G01N 2333/70514; G01N 2333/70596; G01N 2333/96436

(57) ABSTRACT

The present invention relates to a method of predicting, assessing or monitoring the sensitivity of a subject having a cancer to an immunotherapy, preferably to an immunotherapy combining at least two immunotherapeutic agents, and to corresponding kits and uses thereof. The method of predicting, assessing or monitoring the sensitivity of a subject having a tumor to an immunotherapy typically comprises a step of assessing, before any immunotherapeutic treatment step in the subject, the presence of CD4+CD25highCD39high T cells in a tumor sample of the subject, the presence of CD4+CD25highCD39high T cells in the tumor sample of the subject being indicative of sensitivity of the subject to the immunotherapy, and the absence of CD4+CD25highCD39high T cells in the tumor sample of the subject being indicative of resistance of the subject to the immunotherapy.

### FIELD OF THE INVENTION

The present invention relates to a method of predicting, assessing or monitoring the sensitivity of a subject having a cancer or malignant tumor to immunotherapy, and to corresponding kits. The method of predicting, assessing or monitoring the sensitivity of a subject having a cancer or malignant tumor to a proposed immunotherapy typically comprises a step a) of determining, in a biological sample from said subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a tumor is responsive or resistant to the proposed immunotherapy.

### BACKGROUND OF THE INVENTION

Combination immunotherapy, in particular using anti-PD1 and anti-CTLA4 monoclonal antibodies, is a standard of care in first line for the treatment of patients suffering of cancer, in particular of advanced melanoma.

This Combination immunotherapeutic treatment targets immune checkpoints expressed by T-cells in order to invigorate the patients' immune response against his/her cancer. But those treatments can also generate auto-immune and inflammatory responses against healthy tissues (immune related adverse events, “irAE”).

Those immunotherapies generate high response rates, durable remissions and overall survival benefits compared to historical conventional melanoma therapies (Larkin et al., 2019). However, as an example, about 45% of advanced melanoma patients do not respond to those treatments. Also, those treatments induce a high rate of treatment related adverse events which sometime could be fatal (˜1% of patients). Moreover, those therapies are very expensive for health insurances and social securities.

Routine tumor biopsies are either immediately fixed or frozen and used for:
histological diagnostic with formalin fixed paraffin embedded histology & immuno histo chemistry (IHC) stainings (e.g PD-L1 stainings) (Ilie et al., 2016),
DNA and RNA sequencing (e.g BRAF/MEK mutations) (Wagle et al., 2011).

All the rationale for anti-PD1 and anti-CTLA4 immunotherapies has been focused so far on the cytotoxic response of CD8⁺ T-cells and more recently on tertiary lymphoid structures (TLS) and B-cell responses (cf. Tumeh et al., 2014; Vanhersecke et al., 2021).

Inventors now advantageously herein provide a new diagnostic tool to identify patients who can respond and benefit from combined immunotherapies, in particular of an anti-PD1 and anti-CTLA4 combination therapy, in particular a nivolumab (“Nivo”) and ipilimumab (“Ipi”) combination therapy.

### SUMMARY OF THE INVENTION

Personalized therapy of cancer currently relies on the identification of drug targetable tumor cell autonomous signaling pathways. However, immunomodulation of the tumor microenvironment may also be amenable to a more personalized management, and predictive tools for this decision making are awaited.

Inventors herein identify predictive biomarkers that are able to secure identification of cancer patients proned to respond or, on the contrary, resist to a proposed immunotherapy or combined immunotherapies, for example the combination of an anti-PD-1 monoclonal antibody and of an anti-CTLA4 antibody. The present invention includes methods and kits for predicting, assessing or monitoring the response of a subject having cancer (herein equivalent to “malignant tumor”) to a particular immunotherapeutic (combination) treatment using these biomarkers.

A first method herein described is an in vitro or ex vivo method of predicting, assessing or monitoring the sensitivity of a subject having a cancer to a proposed immunotherapy, preferably to an immunotherapy combining at least two immunotherapeutic agents, in particular an anti-PD-1 monoclonal antibody and an anti-CTLA4 antibody.

The method typically comprises a step of determining, before any immunotherapeutic treatment step in the subject, in a sample of the subject, preferably a sample comprising tumor cells, typically a tumor sample of the subject, the presence of CD4⁺CD25highCD39high T cells, the presence of CD4⁺CD25highCD39high T cells in the tumor sample being indicative of sensitivity of the subject to the immunotherapy, and the absence of CD4+CD25highCD39high T cells in the tumor sample being indicative of resistance of the subject to the immunotherapy.

The method may in another aspect comprise a step a) of determining, in a biological sample from said subject, preferably a sample comprising tumor cells, typically a tumor sample of the subject, the presence, absence or expression level of at least one biomarker, for example at least two biomarkers, and when the expression level is determined a step b) of comparing said expression level to reference expression level(s) or to reference expression ratio(s), thereby predicting, assessing or monitoring whether the subject having a tumor is responsive or resistant to the proposed immunotherapy. A particular in vitro or ex vivo method of predicting, assessing or monitoring the sensitivity or resistance of a subject having a cancer to an immunotherapy is performed after one or several treatment steps/cycles with said immunotherapy in the subject, for example after the first cycle and before the second cycle. Such a method preferably comprises a step a) of determining, in a biological sample, preferably a tumor sample of the subject, the expression level of CD4⁺CD25highCD39high T cells, and a step b) of comparing said CD4⁺CD25highCD39high T cells level to a CD4⁺CD25highCD39high T cells reference expression level, thereby predicting, assessing or monitoring whether the subject having a cancer is responsive or resistant to the immunotherapy. Typically, an expression level of CD4⁺CD25highCD39high T cells below the CD4⁺CD25highCD39high T cells reference expression level is indicative of sensitivity of the subject to the immunotherapy and an expression level of CD4⁺CD25highCD39high T cells above the CD4⁺CD25highCD39high T cells reference expression level is indicative of resistance of the subject to the immunotherapy.

Another method herein described is an in vitro or ex vivo method of predicting, assessing or monitoring the sensitivity of a subject having a cancer to a proposed immunotherapy, preferably to an immunotherapy combining at least two immunotherapeutic agents, in particular an anti-PD-1 monoclonal antibody and an anti-CTLA4 antibody using Granzyme B and/or Granzyme A as biomarker(s). This method comprises a step of determining the concentration of Granzyme B and/or Granzyme A in a tumor secretome sample from the subject at baseline, a concentration of Granzyme B and/or Granzyme A in the subject's sample above a reference level, such as the level of Granzyme B and/or Granzyme A in a population of subjects known as being non-responders, being indicative that the subject is sensitive to the immunotherapy, and a concentration of Granzyme B and/or Granzyme A in the subject's sample below a reference level, such as the level of Granzyme B and/or Granzyme A in a population of subjects known as being non-responders, being indicative that the subject is resistant to the immunotherapy.

An additional method herein described is an in vitro or ex vivo method of predicting, assessing or monitoring the sensitivity of a subject having a cancer to a proposed immunotherapy, preferably to an immunotherapy combining at least two immunotherapeutic agents, in particular an anti-PD-1 monoclonal antibody and an anti-CTLA4 antibody using one or several biomarkers selected from Granzyme B, VEGF (A), IL6 and CXCL8 (IL8) as biomarker(s). This method comprises a step of determining the concentration of one or several biomarkers selected from Granzyme A or B, VEGF (A), IL6 and CXCL8 (IL8) as biomarker(s) in a tumor secretome sample from the subject at baseline, a concentration of any of said biomarker(s) in the subject's sample above a reference level, such as the level of said biomarker(s) in a population of subjects known as being non-responders, being indicative that the subject is sensitive to the immunotherapy, and a concentration of said biomarker(s) in the subject's sample below a reference level, such as the level of said biomarker(s) in a population of subjects known as being non-responders, being indicative that the subject is resistant to the immunotherapy.

In a particular aspect, the step of determining the concentration of one or several biomarkers selected from Granzyme A or B, VEGF (A), IL6 and CXCL8 (IL8) as biomarker(s) is performed a) in a tumor secretome sample from a subject/patient suffering of cancer at baseline, i.e. on the diagnosis stage, before any treatment of the subject with a combination of immunotherapeutic agents, for example before any treatment with an anti-CTLA4 and/or anti-PD1 agents, or in a subject/patient who has been exposed to a first treatment of cancer, for example to a chemotherapy, but has not been exposed a combination of immunotherapeutic agents, and b) in a secretome sample from said subject/patient after exposition of the subject to a combination of at least two immunotherapeutic agents, in particular an anti-PD-1 monoclonal antibody and an anti-CTLA4 antibody, and the concentrations obtained in steps a) and b) are then compared, a concentration of any of said biomarker(s) obtained in step b) identical or below the concentration of the biomarker(s) obtained in step a) being indicative that the subject is sensitive to the immunotherapy, and a concentration of any of said biomarker(s) obtained in step b) above the concentration of the biomarker(s) obtained in step a) being indicative that the subject is resistant to the immunotherapy.

Another particular method herein described is a method of selecting an appropriate therapeutic treatment for a subject, which method comprises a step of predicting or assessing the sensitivity of a subject having a cancer or a malignant tumor to an immunotherapy, preferably to an immunotherapy combining at least two immunotherapeutic agents, in particular an anti-PD-1 monoclonal antibody and of an anti-CTLA4 antibody, using a method according to the present invention as described herein above. If the subject is identified as resistant to the proposed immunotherapy, the method further advantageously comprises an additional step of selecting a distinct therapeutic treatment of cancer more appropriate for the subject.

Also herein described is a method for screening or identifying a compound suitable for improving the treatment of a cancer in a subject having a cancer or a malignant tumor, said method comprising determining the ability of a test compound to modify the expression of at least one of the herein described biomarkers of response or resistance to immunotherapy, or compensate an abnormal expression thereof.

Further herein described is a method of selecting or disqualifying a subject having a cancer for inclusion in a clinical trial, the clinical trial being for evaluating an immunotherapy directed against a cancer, preferably an immunotherapy combining at least two immunotherapeutic agents, in particular an anti-PD-1 monoclonal antibody and an anti-CTLA4 antibody, which method comprises a step of predicting or assessing the sensitivity of a subject having a cancer to an immunotherapy combining at least two immunotherapeutic agents using a method according to the present invention as described herein above.

A further embodiment relates to the use of a kit for predicting, assessing or monitoring the sensitivity of a subject having a cancer or a malignant tumor to a cancer therapy, wherein the kit comprises, as detection means, possibly in suitable container means, at least two agents, each of said agents specifically recognizing one of the herein described biomarkers. These at least two agents are typically at least two antibodies selected from the group consisting of an antibody specific to CD4, CD25 and CD39, and optionally to CD3, CD45 and/or CD127, and, optionally, a leaflet providing reference expression levels corresponding to said protein(s).

In a particular aspect, the kit comprises at least one additional agent specifically recognizing (/capable of detecting) one of ICOS, OX40, 41BB (CD137), 42BB, TIGIT, CTLA-4 and Granzyme A or B.

The kit may also comprise a positive control or several positive controls that can be used to determine whether a given agent is capable of specifically recognizing its corresponding biomarker. The kit may also include other reagents that allow visualization or other detection of anyone of the herein described biomarkers, such as reagents for colorimetric or enzymatic assays.

### DETAILED DESCRIPTION OF THE INVENTION

Inventors herein identify predictive biomarkers that are able to secure identification of cancer patients proned to respond or resist to a proposed immunotherapy, preferably an immunotherapy combining at least two immunotherapeutic agents, in particular an anti-PD-1 monoclonal antibody and an anti-CTLA4 antibody.

Inventors performed a clinical trial of anti-PD1 and anti-CTLA4 combination in first line advanced melanoma. They found that all the patients presenting with a specific subpopulation of T-cells identified by several expression markers from the list comprising CD45, CD3, CD4, CD39 and CD25, in particular identified by the presence at least of the three following biomarkers: CD4, CD39 and CD25, in their tumor biopsy at baseline (further herein identified “39/25T4”) responded to the combination treatment of intravenous anti-PD1 and intravenous or intratumoral anti-CTLA4 (cf. FIG. 1A).

Indeed, in the translational research part of the NIVIPIT trial, inventors depicted an interesting intratumoral immune cell population. Patients having a high proportion of CD4⁺CD25highCD39high at baseline (i.e. on the diagnosis stage and/or before any treatment of cancer, in particular before any treatment with a combination of immunotherapeutic agents, for example before any treatment with an anti-CTLA4 and/or anti-PD1 agents) were keener to present a tumor response and a durable clinical benefit (“DCB”). Patients identified as having a DCB are patients keeping an objective tumor response per RECIST1.1 criteria (i.e., either a complete or partial response) to the immunotherapeutic treatment (“CR” or “PR”) at 6 months or exhibiting stable disease at 6 months after the start of immunotherapy (“SD”). These DCB patients are to be distinguished from patients exhibiting progressive disease (“PD”) before six months (also herein identified as “NDCB” or “No DCB” patients). Furthermore, on-treatment biopsies showed a decrease of this CD4⁺CD25highCD39high T cells sub-population among responders, while not in non-responders.

Interestingly enough in the NIVIPIT study, inventors noticed a similar distribution and evolution of CD4⁺CD25highCD127low cells (patients analyzed using a different flow cytometry panel) under treatment, thus suggesting that this population was consistent with intratumoral Tregs.

Parallel experiments on fresh tumor samples (surgical specimens), allowed for flow cytometry using panels containing both anti-CD4, anti-CD25 and anti-CD39, as well as anti-FoxP3 antibodies in order to further support the hypothesis that CD4⁺CD25highCD39high are activated intratumoral Tregs (FIG. 1A).

Single cell RNA-seq data were also used in order to compare at a transcriptomic level, the gene expression coding for CD4, FoxP3, CD25 and CD39 but also activation factors such as ICOS, OX40, 41BB or CTL14 (FIG. 1B).

Data coming from 7 melanoma patients analyzed using flow cytometry and from 2 non-small lung cancer (NSCLC) and 1 head and neck squamous cell carcinoma (HNSCC) analyzed with scRNA-seq, were concordant with the observation that CD4+CD25highCD39high cells, express either the gene or the protein in a very high percentage (>75%), and coactivation markers, hence confirming an activated “Treg” like phenotype. These data have been confirmed by inventors also in Malignant Mesothelioma (MM), Renal Cell Carcinoma (RCC), Epithelial Ovarian Cancer (EOC) and Urothelial Carcinoma (UC).

Inventors now herein reveal for the first time that the presence of a particular subset of CD4+ T-cells, which preferably co express the activation markers CD39 and CD25 (also herein identified as “39/25T4s” T cells) in tumors at baseline (i.e. on the diagnosis and/or before any treatment of cancer, in particular before any treatment of cancer with a combination of immunotherapeutic agents, for example before any treatment of cancer with anti-CTLA4 and/or anti-PD1 agents) can be used as an efficient biomarker to predict response to a combination therapy involving at least two immunotherapeutic agents, in particular an anti-PD1 and an anti-CTLA4 agents.

The subpopulation of CD4+CD25highCD39high T-cells has never been associated to the efficacy of a combination of immunotherapies, for example to the efficacy of a combined anti-PD1+anti-CTLA4 immunotherapy, before the present disclosure. Biomarkers of response reported for anti-PD1 and anti-CTLA4 included so far PD-L1 (as expressed in the tumor and determined by IHC according to Kerr et al., 2015), tumor infiltrates with CD3⁺ or CD8⁺ T-cells (but not CD4⁺ T-cells) as determined IHC according to Tumeh et al., (2014) (IHC stainings being not appropriate for CD4⁺ T cells), and Tumor Mutational Burden (TMB) as determined by DNA sequencing (Marabelle et al., 2020).

The new biomarker consisting of CD4+CD25highCD39high T-cells found in the tumor of the subject at baseline allows to select for such therapeutic option only the patients who could benefit from a combination of immunotherapies, for example of a combination of anti-PD1 and anti-CTLA4 immunotherapies. Therefore, the present invention advantageously allows for the first time to:
prescribe those treatments only to patients who will benefit from them:
avoid to expose patients to severe adverse events if they have no chances of disease response to these treatments;
avoid costly prescriptions in 45% of patients who will not benefit from the treatment;
stratify patient care and help physicians and companies to design new clinical trials and treatment strategies for patients who either express (presence of the CD4+CD25highCD39high T-cells biomarker/“39/25T4 positive”) or do not express (absence of the CD4+CD25highCD39high T-cells biomarker/“39/25T4 negative”) this biomarker;
stratify patients at risk in randomized trials; and
identify a new patient population with specific needs of new treatments.

A first object of the invention is an in vitro or ex vivo method of predicting, assessing or monitoring the sensitivity of a subject having a cancer to a proposed immunotherapy, preferably to an immunotherapy combining at least two immunotherapeutic agents, in particular an anti-PD-1 monoclonal antibody and an anti-CTLA4 antibody.

The method typically comprises a step of determining, before any immunotherapeutic treatment step in the subject, in a sample of the subject, preferably a sample comprising tumor cells, typically a tumor sample of the subject, the presence of CD4⁺CD25highCD39high T cells, the presence of CD4⁺CD25highCD39high T cells in the tumor sample being indicative of sensitivity of the subject to the immunotherapy, and the absence of CD4+CD25highCD39high T cells in the tumor sample being indicative of resistance of the subject to the immunotherapy.

In a particular aspect, the CD4+CD25highCD39high T cell further expresses at least one additional marker selected from CD3, CD45, CD127 and Foxp3.

In another particular aspect, the CD4⁺CD25highCD39high T cell is a CD127low T cell.

The method typically comprises a step a) of determining, in a biological sample from said subject, preferably a sample comprising tumor cells, typically a tumor sample of the subject, the presence, absence or expression level of at least one biomarker, for example at least two biomarkers, and, when the expression level is determined, a step b) of comparing said expression level to reference expression level(s) or to reference expression ratio(s), thereby predicting, assessing or monitoring whether the subject having a cancer is responsive or resistant to the proposed immunotherapy.

The immunotherapy (also herein identified as “chemotherapeutic drug” or “chemotherapeutic agent”) is typically selected from an antibody, preferably a monoclonal antibody, a chemokine and a cytokine. In a preferred aspect, the immunotherapy is an immunotherapy combining at least two immunotherapeutic agents, for example two monoclonal antibodies, in particular an anti-PD-1 monoclonal antibody and an anti-CTLA4 antibody.

The at least two monoclonal antibodies can be advantageously selected from anti-PD-1 monoclonal antibody, anti-PD-L1 (ligand) monoclonal antibody, anti-CTLA-4 monoclonal antibody, anti-CD137 monoclonal antibody, and anti-CD137L (ligand) monoclonal antibody. Preferably, the anti-PD-1 monoclonal antibody(ies) is/are selected from nivolumab, pembrolizumab and a combination thereof.

Relevant examples of anti-PD-1 monoclonal antibodies are nivolumab (BMS-936558, MDX-1106 or ONO-4538, Bristol-Myers Squib), pembrolizumab, also known as lambrolizumab (MK-3475, Merck), pidilizumab (formerly CT-011, CureTech Ltd). Preferred examples are nivolumab and pembrolizumab.

Relevant examples of anti-PD-L1 monoclonal antibodies are atezolizumab (MPDL 3280A, Genentech), BMS 936559 or MDX-1105 (Bristol-Myers Squibb), durvalumab (MEDI4736, MedImmune LLC), avelumab (MSB0010718C, Merck Serono). A preferred example is atezolizumab.

Relevant examples of anti-CTLA-4 monoclonal antibodies are ipilimumab (Yervoy or MDX-010 Bristol-Myers Squibb), tremelimumab (formerly ticilimumab or CP-675,206, Pfizer). Preferred examples are ipilimumab and tremelimumab. Preferably, the anti-CTLA4 monoclonal antibody (ies) is/are selected from nivolumab, pembrolizumab and a combination thereof. Relevant examples of anti-CD137 monoclonal antibodies is urelumab (BMS-663513, Bristol-Myers Squibb). A preferred example is urelumab.

Cytokines can be selected from pegylated interferon alpha 2a and alpha 2b, IL-2 (proleukin) and IL-2/mAb complexes (also termed IL-2 complexes or IL-2/anti-IL-2 mAb complexes consisting of IL-2 associated to a particular anti-IL-2 mAb). The cytokine is preferably selected from IFNα2α (ROF) and IL-2.

In a preferred embodiment of the invention, the immunotherapy is a combined treatment. Preferred combined immunotherapy is a combination of anti-PD-1 monoclonal antibody and of an anti-CTLA-4 monoclonal antibody. Other combined immunotherapies can involve anti-KIR, anti-OX40, anti-ICOS, anti-VISTA, anti-TIGIT, anti-CD96 and/or anti-BTLA, for example. In the present invention, the cancer is a cancer that is usually or conventionally treated with one of the herein above described immunotherapy, preferably with an anti-CTLA-4 monoclonal antibody, with an anti-PD-1 monoclonal antibody or with a combination thereof.

The cancer or tumor is typically selected from melanoma, lung, in particular non-small cell lung cancer or small cell lung cancer (NSCLC), head and neck cancer, in particular Head and Neck Squamous Cell Carcinoma ((HNSCC), bladder cancer, in particular a bladder cancer with lymph nodes (LN) metastasis, Urothelial Carcinoma (UC), mesothelioma cancer (“Malignant Mesothelioma” or “MM”), oesophagus cancer, stomach cancer, hepatocarcinoma cancer, kidney or renal cancer, breast cancer, in particular triple negative breast cancer, Epithelial Ovarian Cancer (EOC), and more generally any cancer amenable to immune checkpoint blockade or leading to stimulation of the immune system.

In a particular aspect, the cancer is selected from melanoma, lung, head and neck cancer, renal cancer and bladder cancer, and is preferably a melanoma, in particular an advanced or a stage III or IV melanoma.

In a preferred embodiment the cancer is a melanoma, in particular an advanced melanoma or a stage III or a stage IV melanoma, typically a stage IV melanoma affecting at least skin and LN. The cancer or tumor is preferably selected from melanoma, lung, renal cancer, head and neck cancer, bladder cancer, and is even more preferably a melanoma, in particular a stage III or stage IV melanoma.

In the context of the present invention, the patient or subject is a mammal. In a particular embodiment, the mammal is a human being, whatever its age or sex. The patient typically has a tumor. Unless otherwise specified in the present disclosure, the tumor is a cancerous or malignant tumor. Preferably the subject is a subject who has not been previously exposed to a treatment of cancer, or a subject who has received a chemotherapeutic drug but who has not been treated with an immunotherapy in particular with an immunotherapy combining at least two immunotherapeutic agents, for example an anti-PD-1 monoclonal antibody and an anti-CTLA4 antibody.

In a preferred embodiment of the present invention, the method of the invention is performed after at least partial, for example total, resection of the cancerous tumor and/or metastases thereof, in the subject. The method can however also be performed on the subject before any surgical step.

In the context of melanoma cancer, a particular subpopulation of subjects is composed of stage III or stage IV melanoma, typically a subpopulation of subjects having undergone at least partial tumor resection. Another particular subpopulation of subjects is composed of subjects having metastases.

In the context of lung cancer, a particular subpopulation of subjects is composed of locally advanced, non operable non small cell lung cancer (NSCLC), or metastatic lung cancer. Another particular subpopulation of subjects is composed of subjects having lung cancer, in particular NSCLC, and metastases.

A particular method of predicting, assessing or monitoring the sensitivity of a subject having a cancer to a proposed immunotherapy according to the invention comprises a step a) of determining, in a biological sample from said subject, preferably a sample comprising tumor cells, typically a tumor sample of the subject, the presence, absence or expression level of at least one biomarker, for example at least two biomarkers, for example one or several biomarkers selected from Granzyme A or B, VEGF (A), IL6 and CXCL8 (IL8) as biomarker(s), and, when the expression level is determined, a step b) of comparing said expression level to reference expression level(s) or to reference expression ratio(s), thereby predicting, assessing or monitoring whether the subject having a tumor is responsive or resistant to the proposed immunotherapy.

A particular in vitro or ex vivo method of predicting, assessing or monitoring the sensitivity or resistance of a subject having a cancer to an immunotherapy is performed after one or several treatment steps/cycles with said immunotherapy in the subject. Such a method preferably comprises a step a) of determining, in a biological sample, preferably a tumor sample of the subject, the expression level of CD4⁺CD25highCD39high T cells, and a step b) of comparing said CD4⁺CD25highCD39high T cells level to a CD4+CD25highCD39high T cells reference expression level, thereby predicting, assessing or monitoring whether the subject having a cancer is responsive or resistant to the immunotherapy. Typically, an expression level of CD4⁺CD25highCD39high T cells below the CD4⁺CD25highCD39high T cells reference expression level is indicative of sensitivity of the subject to the immunotherapy and an expression level of CD4⁺CD25highCD39high T cells above the CD4⁺CD25highCD39high T cells reference expression level is indicative of resistance of the subject to the immunotherapy.

In a particular aspect, the CD4+CD25highCD39high T cell further expresses at least one additional marker selected from CD3, CD45, CD127 and Foxp3.

In another particular aspect, the CD4⁺CD25highCD39high T cell is a CD127low T cell.

Implementations of the methods of the invention involve obtaining a (biological) sample from a subject. The sample can be a fluid sample and may include any specimen containing immune cells such as blood, lymphatic fluid, spinal fluid, pleural effusion, ascites, or a combination thereof. The biological sample is preferably a sample comprising tumor cells. Such a sample can be a tumor biopsy, a whole tumor piece, a tumor bed sample, a metastatic lymph node cells sample, or a combination thereof. The sample is preferably a fresh tumor sample biopsy or a tumor sample biopsy which has not been frozen. In a particular aspect, the fresh tumor sample biopsy or tumor sample biopsy which has not been frozen, is dissociated with both enzymatic and mechanical procedures before being stained and used in the context of a herein described method or use.

The method of the invention preferably comprises a step of dosing via ELISA at least one marker selected from VEGFA, IL6, CXCL8, granzyme, in the supernatant of a fresh tumor sample biopsy after an incubation step of at least one minute, for example after an incubation step of between 1, 2 or 3 minutes and 40 minutes, preferably at least 30 minutes.

By “sensitivity” or “responsiveness” is intended herein the likelihood that a patient will respond to a chemotherapeutic treatment as herein described.

By “resistant” is intended herein the likelihood that a patient will not respond to such a chemotherapeutic treatment.

Predictive methods of the invention can advantageously be used clinically to make treatment decisions by choosing as soon as possible the most appropriate treatment modalities for a particular patient and limit toxicities classically associated to immunotherapy.

If the subject is identified, using a method according to the present invention, as resistant to a particular treatment of cancer, the method advantageously further comprises a step of selecting a distinct cancer treatment, for example a distinct immunotherapy typically involving a “compensatory molecule” to be used alone or in combination with the originally preselected (chemo) therapeutic drug(s) or with (a) distinct (chemo) therapeutic drug(s), as the appropriate therapeutic treatment of cancer for the subject.

Preferably, the step of determining the presence, absence or expression level of at least one biomarker, for example at least two or three biomarkers, in a biological sample of the subject is performed before any cancer therapy, typically immunotherapeutic treatment step.

In a particular method of the invention, the at least one biomarker, for example at least two biomarkers, is (are) selected from CD4⁺CD25highCD39high T cells and Granzyme A or B, and the step of determining the presence, absence or expression level of the biomarker(s) in a biological sample, preferably biological sample comprising tumor cells, even more preferably tumor biopsy, of the subject is performed before any immunotherapeutic treatment step, and optionally after at least partial tumor resection in the subject.

In a particular embodiment, typically when CD4⁺CD25highCD39high T cells are used as a biomarker, this step can be performed three weeks after the first administration (typically injection) of an immunotherapeutic drug (anti-CTLA4 monoclonal Ab, for example ipilimumab) or of a combination of immunotherapeutic drugs (anti-CTLA4 and anti-PD1 monoclonal Ab) to the subject. This step can also be performed after tumor surgical resection.

In a particular embodiment, the method according to the present invention is an in vitro or ex vivo method of assessing, predicting or monitoring the sensitivity of a subject having a melanoma, preferably a stage III or IV melanoma, and the immunotherapy is selected from anti-CTLA-4 monoclonal antibody, anti-PD-1 monoclonal antibody and combination thereof. Herein described is thus an in vitro or ex vivo method of predicting, assessing or monitoring the sensitivity of a subject having a cancer to an immunotherapy selected from anti-PD-1 monoclonal antibody, anti-PD-L1 monoclonal antibody, anti-CTLA-4 monoclonal antibody, and a combination of anti-PD-1 and anti-CTLA-4 monoclonal antibodies, wherein the cancer is a melanoma, in particular a stage III or IV melanoma, and the method comprises a step of determining, before any immunotherapeutic treatment step in the subject, in a tumor sample of the subject, the presence of CD4⁺CD25highCD39high T cells, the presence of CD4⁺CD25highCD39high T cells in the tumor sample being indicative of sensitivity of the subject to the immunotherapy using at least two immunotherapeutic agents. In another aspect, the method is performed after one or several treatment steps, for example one, two, three, four, five, six, seven or eight treatment steps, preferably before beginning any further treatment step, with the immunotherapy in the subject, and comprises a step a) of determining, in a tumor sample of the subject, the expression level of CD4⁺CD25highCD39high T cells, and a step b) of comparing said CD4⁺CD25highCD39high T cells level to a CD4⁺CD25highCD39high T cells reference expression level, an expression level of CD4⁺CD25highCD39high T cells below the CD4⁺CD25highCD39high T cells reference expression level being indicative of sensitivity of the subject to the immunotherapy and an expression level of CD4⁺CD25highCD39high T cells above the CD4⁺CD25highCD39high T cells reference expression level being indicative of resistance of the subject to the immunotherapy.

Typically, the expressions “reference value” or “reference expression level” used in the present description is the concentration of the biomarker in a control sample derived from one or more subjects (reference population) having a cancer, and is typically the median value obtained from the reference population. The reference value typically varies in a range of values defined for a given population. The reference value can further be a ratio involving two distinct biomarkers or a % or proportion of one several biomarkers in a control sample.

In a particular and preferred aspect, the CD4⁺CD25highCD39high T cells reference expression level is the level of CD4⁺CD25highCD39high T cells in the tumor of the subject before any immunotherapeutic treatment step in the subject.

In a particular embodiment, when the patient is bearing anyone of a herein described cancer, preferably a melanoma cancer, typically a stage III or IV melanoma, and when the proposed (candidate) immunotherapy is the combination of an anti-PD-1 monoclonal antibody and of an anti-CTLA4 antibody, the method of the invention of predicting, assessing or monitoring the sensitivity of a subject having a cancer to the immunotherapy comprises a step a) of determining, in a tumor sample of the subject, the expression level of CD4⁺CD25highCD39high T cells, and a step b) of comparing said CD4⁺CD25highCD39high T cells level to a CD4⁺CD25highCD39high T cells reference expression level, an expression level of CD4⁺CD25highCD39high T cells below the CD4⁺CD25highCD39high T cells reference expression level being indicative of sensitivity of the subject to the immunotherapy and an expression level of CD4⁺CD25highCD39high T cells above the CD4⁺CD25highCD39high T cells reference expression level being indicative of resistance of the subject to the immunotherapy.

In another particular embodiment, when the patient is bearing anyone of a herein described cancer, preferably a melanoma cancer, typically a stage III or IV melanoma, and when the proposed (candidate) immunotherapy or immunotherapeutic drug is a combination of anti-PD-1 and anti-CTLA-4 monoclonal antibodies, the method of the invention of predicting, assessing or monitoring the sensitivity of a subject having a cancer to the immunotherapy comprises a step a) of determining, in a tumor sample of the subject, the expression level of CD4⁺CD25highCD39high T cells, and/or of determining in a tumor secretome sample of the subject the expression level of one or several biomarkers selected from Granzyme A, Granzyme B, VEGF (A), IL6 and CXCL8 (IL8), in particular of Granzyme B, and a step b) of comparing said level(s) to their respective reference expression level(s), an expression level below for CD4⁺CD25highCD39high T cells, or above for Granzyme A, Granzyme B, VEGF (A), IL6 or CXCL8 (IL8), the reference expression level being indicative of sensitivity of the subject to the immunotherapy, and an expression level above for CD4⁺CD25highCD39high T cells, or below for Granzyme A, Granzyme B, VEGF (A), IL6 or CXCL8 (IL8), the reference expression level being indicative of resistance of the subject to the immunotherapy.

Typically, a cell surface biomarker expression can easily be determined by FACS and a molecule cell release can easily be determined by ELISA as indicated herein above, preferably in the context of the method herein described for the first time by inventors wherein the step of dosing via ELISA at least one marker is performed in the supernatant of a fresh tumor sample biopsy after an incubation step of at least one minute, for example between 1 and 40 minutes, preferably at least 30 minutes, and as further explained below.

In some embodiments of the invention, identification of a biomarker of interest involves use of at least one binding agent. Furthermore, it is contemplated that a binding agent may be specific or not to the considered biomarker. For example, the CD4⁺CD25highCD39high T cells binding agent may bind to a part of CD25 (e.g. an epitope) that is not available depending on whether it is expressed by/bound to CD25⁺CD4⁺ T cells from a biological sample comprising tumor cells as previously described. Alternatively, different conformations may serve the basis for binding agents capable of distinguishing between similar biomarkers.

The binding agent is typically a polypeptide. The polypeptide is, in particular embodiments, an antibody. In further embodiments, the antibody is a monoclonal antibody. The antibody can be bi-specific, recognizing two different epitopes. The antibody, in some embodiments, immunologically binds to more than one epitope from the same biomarker. In some embodiments of the invention, the binding agent is an aptamer.

In some embodiments of the invention, the binding agent is labeled. In further embodiments, the label is radioactive, fluorescent, chemiluminescent, an enzyme, or a ligand. It is also specifically contemplated that a binding agent is unlabeled, but may be used in conjunction with a detection agent that is labeled. A detection agent is a compound that allows for the detection or isolation of itself so as to allow detection of another compound that binds, directly or indirectly. An indirect binding refers to binding among compounds that do not bind each other directly but associate or are in a complex with each other because they bind the same compounds or compounds that bind each other.

When CD4 is to be detected, the antibody to be used can be SK3 (BD Biosciences in PerCP-Reference: 3457703).

When CD25 is to be detected, the antibody to be used can be M-A251 (BD Biosciences in PE-Reference: 555432).

When CD39 is to be detected, the antibody to be used can be A1 (eBiosciences in APC-reference: 15537926).

When CD137L (4-1BBL) is to be detected, the antibody to be used can be C65-485 (BD Biosciences in PE-Reference: 559446).

When CD137 (4-1BB) is to be detected, the antibody to be used can be 4B4-1 (Biolegend-Reference: 309810).

When PD-1 is to be detected, the antibody to be used can be PD1.3.5 (Beckman Coulter in Pe-Cy7—Reference: A78885).

When PD-L1 (CD274) is to be detected, the antibody to be used can be 29E.2A3 (BioLegend in APC-Reference: 329708).

When CD127 is to be detected, the antibody to be used can be MB15-18C9 (Miltenyi Biotec in APC-Reference: 130-094-890).

Other embodiments of the invention involve a second binding agent in addition to a first binding agent. The second binding agent may be any of the entities discussed above with respect to the first binding agent, such as an antibody. It is contemplated that a second antibody may bind to the same of different epitopes as the first antibody. It is also contemplated that the second antibody may bind the first antibody or another epitope than the one recognized by the first antibody.

As discussed earlier, binding agents may be labeled or unlabeled. Any polypeptide binding agent used in methods of the invention may be recognized using at least one detection agent. A detection agent may be an antibody that binds to a polypeptide binding agent, such as an antibody. The detection agent antibody, in some embodiments, binds to the Fc-region of a binding agent antibody. In further embodiments, the detection agent is biotinylated, which is incubated, in additional embodiments, with a second detection agent comprising streptavidin and a label. It is contemplated that the label may be radioactive, fluorescent, chemiluminescent, an enzyme, or a ligand. In some cases, the label is an enzyme, such as horseradish peroxidase.

The present invention also covers methods involving using flow cytometry or ELISA assay to detect biomarkers.

In some embodiments, the selected flow cytometry technology is FACS (Fluorescence-activated cell sorting). FACS can be used for distinguishing and separating into two or more containers specific cells from a heterogeneous mixture of biological cells, based upon the specific light scattering and fluorescent characteristics of each cell.

In other embodiments, the ELISA assay is a sandwich assay. In a sandwich assay, more than one antibody will be employed. Typically, ELISA method can be used, wherein the wells of a microtiter plate are coated with a set of antibodies which recognize the protein of interest. A sample containing or suspected of containing the protein of interest is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In the methods herein described of predicting, assessing or monitoring the sensitivity of a subject having a cancer to an immunotherapy as well as in the methods herein described of selecting an appropriate chemotherapeutic treatment, any classical method known by the skilled person of determining the presence or measuring the expression level of a compound of interest, such as typically FACS, ELISA and radioimmunoassay can be used.

A method of selecting an appropriate, preferably optimal, therapeutic treatment of cancer for a subject having a cancer as herein described, is in addition herein described, as well as appropriate chemotherapeutic treatment involving for example compensatory molecules for use in such a treatment of cancer, possibly in combination with the preselected therapeutic drug(s), typically immunotherapeutic drugs, in a subject identified, using a method as herein described, as resistant to said preselected therapeutic drug(s).

Also herein described is thus a method of selecting an appropriate therapeutic treatment for a subject, which method comprises a step of predicting or assessing the sensitivity of a subject having a cancer or a malignant tumor to a proposed immunotherapy, preferably an immunotherapy combining at least two immunotherapeutic agents, in particular an anti-PD-1 monoclonal antibody and an anti-CTLA4 antibody, using a method according to the present invention as described herein above.

If the subject is identified as sensitive to the proposed immunotherapy, this means that said immunotherapy is an appropriate chemotherapeutic treatment for the subject.

If the subject is identified as resistant to the proposed immunotherapy, this means that said immunotherapy will not be efficient in the subject and will in addition possibly generate unwanted deleterious side effects in the subject. In such circumstances, the method further advantageously comprises an additional step of selecting a distinct or complementary therapeutic treatment of cancer more appropriate for the subject.

For subject suffering of a melanoma, when the combination of an anti-PD-1 monoclonal antibody and of an anti-CTLA4 monoclonal antibody is not efficient, or not efficient alone, in the subject, the distinct therapeutic treatment can be a compound selected from any other immunostimulatory monoclonal antibody such as an antibody targeting TIM3, LAG3, VISTA, BTLA, CD137, OX40, ICOS, B7-H3, B7-H4, KIR, IDO, or TIGIT, and any combination thereof; or a combination of the anti-PD-1 and anti-CTLA4 monoclonal antibodies and of such a distinct compound.

Methods of screening for candidate therapeutic agents for preventing or treating cancer are also included as part of the invention. The method is typically a method which is performed in vitro or ex vivo. When performed ex vivo, it can be performed for example on a sample from a subject who has been administered with a test compound.

A method herein described is typically a method for screening or identifying a compound suitable for improving the treatment of a cancer in a subject having a cancer, said method comprising determining the ability of a test compound to modify the expression of at least one of the herein described biomarkers of response or resistance to immunotherapy, or compensate an abnormal expression thereof.

Further herein described is method of selecting or disqualifying a subject having a cancer for inclusion in a clinical trial, the clinical trial being for evaluating an immunotherapy directed against a cancer, preferably an immunotherapy combining at least two immunotherapeutic agents, in particular an anti-PD-1 monoclonal antibody and an anti-CTLA4 antibody, which method comprises a step of predicting or assessing the sensitivity of a subject having a cancer to an immunotherapy combining at least two immunotherapeutic agents using a method according to the present invention as described herein above.

The present invention also includes kits for predicting, assessing or monitoring the sensitivity of a subject having a cancer or a malignant tumor to a cancer therapy, in particular an immunotherapy, wherein the kit comprises, as detection means, possibly in suitable container means, at least two agents, for example three, four or five agents, each of said agent specifically recognizing one of the herein described biomarkers. These at least two agents are typically at least two antibodies selected from the group consisting of an antibody specific to CD4, CD25 and CD39, and optionally to CD3, CD45 and/or CD127, and, optionally, a leaflet providing the reference expression levels corresponding to anyone of, several or each of said proteins.

In a particular aspect, the kit comprises at least one additional agent specifically recognizing (/capable of detecting) one of ICOS, OX40, 41BB (CD137), 42BB, TIGIT, CTLA-4 and Granzyme A or B.

In further embodiments, the binding agent is labeled or a detection agent is included in the kit. It is contemplated that the kit may include one, at least one or several, biomarker binding agents attached to a non-reacting solid support, such as a tissue culture dish or a plate with multiple wells. It is further contemplated that such a kit includes one or several detectable agents in certain embodiments of the invention. In some embodiments, the invention concerns kits for carrying out a method of the invention comprising, in suitable container means: (a) agent(s) that specifically recognizes all or part of a given biomarker; and, (b) at least one positive control, for example at least two positive controls, that can be used to determine whether the agent is capable of specifically recognizing all or part of said given biomarker. The kit may also include other reagents that allow visualization or other detection of anyone of the herein described biomarkers, such as reagents for colorimetric or enzymatic assays.

The invention will be further illustrated by the following figures and examples. However, these examples and figures should not be interpreted in any way as limiting the scope of the present invention.

### FIGURES

FIG. 1. Flow cytometry and scRNA-seq analysis to caracterize CD4+CD25+CD39+ cells Flow cytometry from 7 melanoma samples showed that CD25+CD39+ cell represented 70.9% (±16.7) of CD4+ cells, and that CD4+CD25+CD39+ cells were also FoxP3+ in 75.7% (±13.4) of cases. These populations expressed significantly more activation molecules such as ICOS, OX40, 42BB, TIGIT or CTLA4 (Mann-Whitney test, p<0.05 for all comparisons but CD4+CD39+ and CD25- or CD25+: p=0.1). On scRNA-seq data: UMAP gene expression clustering from tumor infiltrating lymphocytes (n=3) and violin plot showing log-normalised expression of genes of CD3+CD4+FOXP3+ and CD3+CD4+FOXP3-TILs confirms the significant co-expression of CD4 and Foxp3 gene and genes coding for CD25 and CD39, but also for ICOS, OX40, 41BB (CD137) and CTLA4.

FIG. 2. NIVIPIT trial design scheme

FIG. 3. Safety profile of intratumoral and intravenous arms

Time to Treatment-related grade 3-4 toxicity event-free survival curves (time from randomization to first documentation of treatment-related grade 3-4 toxicity) and incidence at 6 and 12 months (top). Distribution pie charts of treatment related toxicities according to treatment arm (middle). Bar chart showing treatment related grade ≥3 adverse events according to treatment arm (bottom).

FIG. 4. Efficacy results: PFS and OS estimations

Median PFS for the IT arm was 12.2 months [4.4—NR].

Median PFS was not reached for the IV arm nor Median OS for none of the 2 arms. Histograms show the best response rate in the IT and IV arms. Response on injected and non-injected lesions is shown for the IT arm (Waterfall plot).

FIG. 5. Pharmacokinetic results of Ipilimumab and Nivolumab in the IT and IV arms

Violin plots showing plasma levels of Ipilimumab (ELISA assay) and Nivolumab (Liquid chromatography-tandem mass spectrometry technology) in the IT (0.3 mg/kg of Ipilimumab and 1 mg/kg of Nivolumab) and IV arm (3 mg/kg of Ipilimumab and 1 mg/kg of Nivolumab). Blood samples were performed with 30 minutes prior and after immunotherapy administration. Nivolumab was administered after Ipilimumab. The non-parametric Mann-Whitney test was used for comparisons. *<0.5 **<0.01 ***<0.001 ****<0.0001

FIG. 6. Fresh tumor analysis using flow cytometry and secretome titration of responders vs non responders

a. Box plots showing evolution of intratumoral activated Tregs. A higher proportion is observed at baseline, that decrease under treatment for responders.

b. Higher Granzyme A and B levels, that further increase after treatment were observed in responder patients secretome, i.e., the supernatant of tumor biopsies (Meso scale Discovery®). Nota bene: CD4+/CD39high/CD25high and CD4+/CD25high/CD127low were grouped under the appellation “Treg like” (samples were analyzed using 2 different panels during the study) (top). Granzyme A & B concentrations in the supernatant of fresh tumor biopsies of first line stage III & IV melanoma at baseline, with or without Durable Clinical Benefit (DCB) from a combination of anti-PD1 & anti-CTLA4 (bottom).

c. Proportions of CD39+CD25+ cells among CD4+ T-cells at baseline in fresh tumor biopsies of first line stage III & IV melanoma with or without Durable Clinical Benefit (DCB) from a combination of anti-PD1 & anti-CTLA4. Flow cytometry of immune cells performed on fresh biopsy samples shows that at baseline (prior to any treatment), patients having a high proportion of CD4+CD39highCD25high have a significantly higher probability to present a durable clinical benefit (DCB: disease control lasting for more than 6 months).

FIG. 7. Proportions of CD4+FOXP3+ regulatory T-cells (Tregs) in freshly resected primary tumors of different histology (MM: malignant melanoma: NSCLC: Non Small Cell Lung Cancer: RCC: Renal Cell Cancer: HNSCC: Head & Neck Squamous Cell Carcinoma: EOC: Epithelial Ovarian Cancer: UC: Urothelial Cancer).

FIG. 8. Proportions of CD25+ (left), CD39+ (middle) and CTLA4+ (right) cells within CD8+, CD4+Foxp3− et CD4+Foxp3+

FIG. 9. Level of Expression of CD25 and CD39 on CD4+Foxp3− and CD4+Foxp3+ cells A. Level of Expression expressed as mean fluorescent intensity (MFI) of CD25 and CD39 on CD8+, CD4+Foxp3− and CD4+Foxp3+ cells.

B. CD39 and CD25 are the highest checkpoint expressed in terms of Ratio of Mean Fluorescence Intensity (MFI) between Foxp3+ and Foxp3− cells.

FIG. 10. Proportions of CD25+, CD39+ and CTLA4+ cells among CD4+Foxp3+ cells in several tumor types

Respective proportions of CD25+ cells (A), CD39+ cells (B) and CTLA4+ cell among CD4+Foxp3+ cells in several types of tumor. Malignant Mesothelioma (MM), Non-Small Cell Lung Cancer (NSCLC), Renal Cell Carcinoma (RCC), Head and Neck Squamous Cell Carcinoma (HNSCC), Epithelial Ovarian Cancer (EOC) and Urothelial Carcinoma (UC).

FIG. 11. Soluble factor supernatant dosage of biopsies of NIVIPIT secretome (Quantification Score, “QS”)

FIG. 12. Secretome data for biopsies at baseline in NIVIPIT Clinical Trial

FIG. 13. Highly-specific predictive value of CD4+CD25highCD39high cells

A ROC curve analysis was done using n=19 patients with CD4+CD25highCD39high frequencies as a predictor of event (death). The resulting AUC of the ROC curve demonstrates a highly-specific and moderately-sensitive predictive value of CD4+CD25highCD39high frequencies.

B. Kaplan Meier curves of OS in patients with CD4+CD25highCD39high <3.61 (gray) and CD4+CD25highCD39high ≥3.61 (black). Statistical comparison was done using the Log-Rank test.

FIG. 14. Highly-specific predictive value of “low” secretome Granzyme A and Granzyme B

A. ROC curve analysis was done using n=19 patients with Granzyme A frequencies as a predictor of event (death)

B. ROC curve analysis was done using n=20 patients with Granzyme B frequencies as a predictor of event (death).

C. Kaplan Meier curves of OS in patients with Granzyme A high (gray) ≥16O pg/ml and Granzyme A low ≤160 pg/ml (black). Statistical comparison was done using the Log-Rank test. 5D. Kaplan Meier curves of OS in patients with Granzyme B high (gray) ≥11.6 pg/ml and Granzyme B low ≤11.6 pg/ml (black). Statistical comparison was done using the Log-Rank test.

Throughout this application, various references describe the state of the art to which this invention pertains. The disclosures of these references are hereby incorporated by reference into the present disclosure.

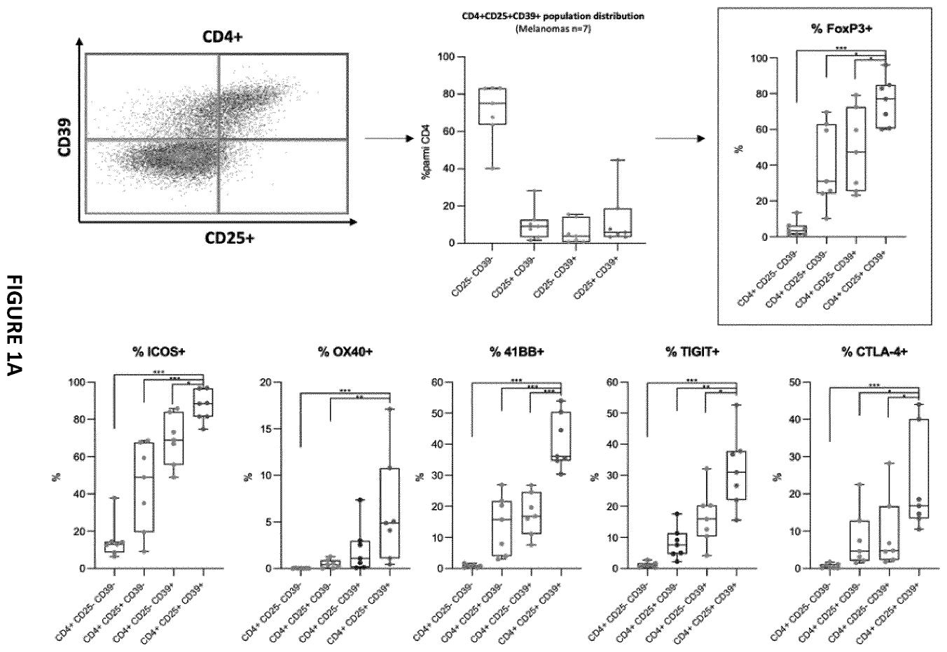

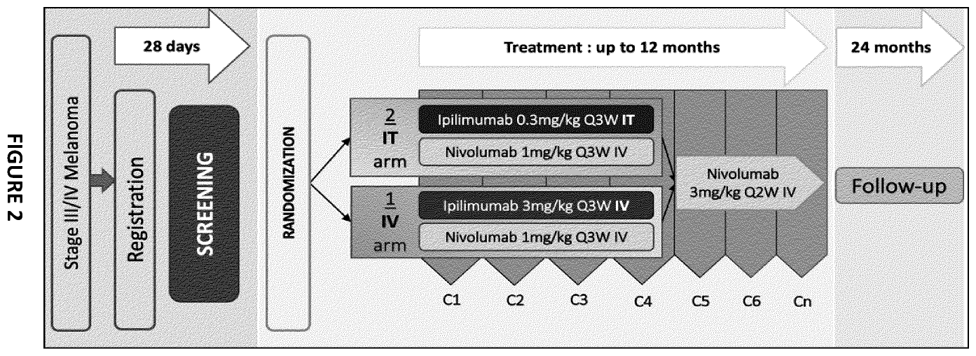

Other characteristics and advantages of the invention are given in the following experimental section (with reference to FIGS. 1 to 6), which should be regarded as illustrative and not limiting the scope of the present application.

### EXPERIMENTAL PART

### Example 1—Safety and Efficacy of Intravenous (IV) Nivolumab with Intratumoral (IT) Ipilimumab in Metastatic Melanoma

Inventors herein below describe the results of the NIVIPIT trial, a randomized multicenter Phase 1b study comparing the intratumoral (IT) administration of Ipilimumab (“Ipi”), an IgGI anti-CTLA4, to the intravenous (IV) Ipi with intravenous (IV) Nivolumab (“nivo”) administration, in patients (Pts) with Metastatic Melanoma (“MM”).

### Methods

This study was approved by the national ethics committee.

Previously untreated metastatic melanoma patients (n=61) (Table 1 Patient Characteristics) were randomly assigned 1:2, to receive IV Nivo (1 mg/kg) in combination with either IV Ipi (3 mg/kg) (IV arm: n=21) or 10× lower dose IT Ipi (0.3 mg/kg) (IT arm: n=40 every 3 weeks for 4 doses), followed by Nivo 3 mg/kg every 2 weeks for up to 12 months (FIG. 2).

TABLE 1
Patient Characteristics

| | IT Arm | IV Arm |
|---|---|---|
| | N = 40 | N = 21 |
| Sex, n (%) | | |
| Male/Female | 26 (65%)/14 (35%) | 12 (57%)/9 (43%) |
| Age at randomization | | |
| Median: years old [range] | 55 [22-79] | 58 [28-82] |
| Site, n (%) | | |
| Gustave Roussy, Villejuif | 21 (53%) | 9 (43%) |
| IC-HCL, Lyon | 14 (35%) | 9 (43%) |
| Saint-Louis APHP, Paris | 4 (10%) | 2 (10%) |
| IUCT Oncopole, Toulouse | 1 (3%) | 1 (5%) |
| BRAF status, n (%) | | |
| Wild type | 21 (53%) | 13 (62%) |
| Mutated | 19 (47%) | 8 (38%) |
| AJCC Stage, n (%) | | |
| IlIC | 0 | 1 (5%) |
| IIID | 6 (15%) | 2 (10%) |
| IV | 34 (85%) | 18 (86%) |
| LDH, n (%) | | |
| < ULN (upper limit normal) | 19 (53%) | 10 (53%) |
| > ULN | 17 (47%) | 9 (47%) |
| missing | 4 | 2 |
| NLR (neutrophil/lymphocyte) | | |
| Median: [range] | 2.4 [1.4-20.5] | 3.3 [1.1-6.9] |

Flow Cytometry Immune Cells Phenotyping and Analysis from tumor biopsies

Core biopsy samples from tumors at baseline and prior to cycle 2 (injected and non-injected) were immediately placed into 1 ml of NaCl 0.9% and sent to the laboratory (LRTI-U1015). After a minimum of 30 minutes of incubation, fine-needle biopsies were mechanically dissociated with the bottom of a 2 ml syringe in a wet 70 μm filter placed at the top of a 50 ml centrifuge tube. Isolated cells were then washed by centrifugation and the pellet was re-suspended in an appropriate volume of NaCl 0.9% for cell surface staining protocol. Antibodies mix was composed of immune markers CD45, CD3, CD4, CD8 and HLA-ABC, activation markers HLA-DR and CD25, T-Regulator markers CD39 and CTLA-4, Immune checkpoint markers PD-1, OX40 and TIGIT and a co-stimulator marker CD26. CTLA-4 was first stained at +37° C. for 20 min before others surface antibodies were added and incubated at +4° C. for 15 min. Two different panels were used, as CD39, CD26 and CD33 have been added, and CD127 and 41BB have been abandoned after the first 9 patients (for 22 patients). Then, cells were washed twice and acquisition were performed on an 18-colors flow cytometer BD Fortessa X20 (BD Biosciences). Data were processed in FCS 3.0 format and analyzed with KALUZA software version 2.1. From our population of interest, doublets were first excluded based on forward-scatter-Height versus forward-scatter Area plot, and then viable cells were selected. Tumor infiltrating T-lymphocytes were then selected with a CD45+ and CD3+ gate, and then were divided into two sub-populations based on CD4 and CD8 expression.

### Plasmatic and Secretome Cytokines Measurement

To evaluate soluble factors in patient's plasma and/or supernatant of biopsies, a Quickplex SQ120 platform enabling highly sensitive electro-chemo-luminescent detection (Meso Scale Discovery, Rockville, MD) was used following the manufacturer's instructions. The analytes measured were: Interleukins 6 (IL-6), 8 (IL-8): Vascular Endothelial Growth Factor (VEGF); Enzymatic proteins (Granzyme A and B): . Absolute concentrations of soluble analytes (in pg/mL or fg/mL) in patient samples were calculated by use of a four-point-fit calibration curve of the standard dilutions (MSD DISCOVERY WORKBENCH analysis software) and were considered “detectable” if both runs of each sample had a signal greater than the analyte- and plate-specific lower limit of detection (LLOD).

The primary objective was to compare ≥grade 3 immune related Adverse Events (“irAE”) rates at 6 months.

Secondary objectives were safety and efficacy evaluation of the IT arm and to explore predictive immune biomarkers on blood and tumor samples using flow cytometry and chemokine/cytokines titration.

Fresh tumor biopsies pre- and on-treatment on both injected and non-injected tumors were analyzed by flow cytometry, and soluble factors from their supernatant (“secretome”) were titrated with Meso Scale Discovery R multiplex (cf. FIG. 1A).

Fresh sequential whole blood samples were collected for flow cytometry phenotyping of immune cells, and for measuring systemic exposure to Ipi (PK) using ELISA (cf. FIG. 1B).

### Results

40 patients were treated in the IT arm and 21 in the IV arm.

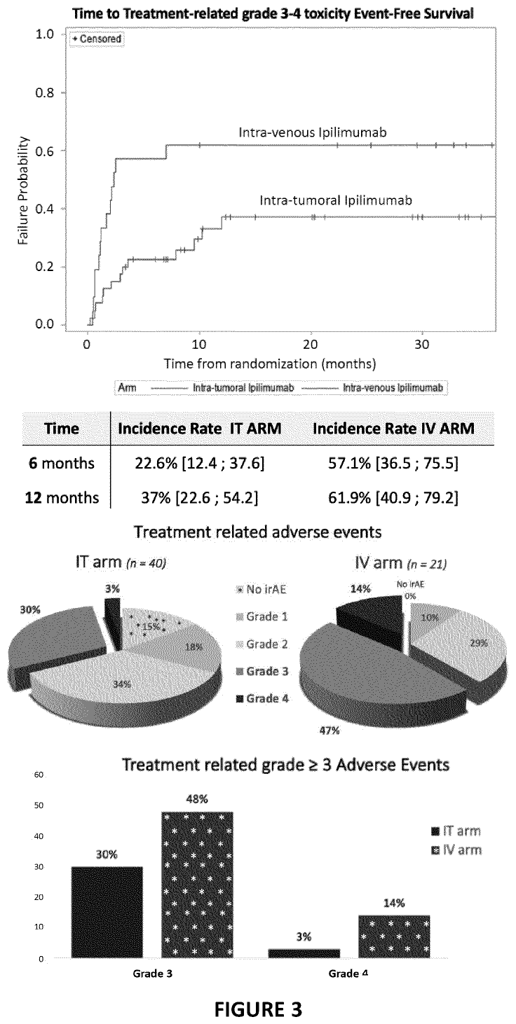

The study met its primary endpoint with lower toxicity rate at 6 months in the IT arm, with 22.6% [12.4:37.6] vs 57.1% [36.5:75.5] of patients presenting ≥grade 3 treatment related Adverse Events (“AEs”), and no procedure-related ≥grade 3 AEs in the IT arm out of 162 IT injections performed (including deep seated lesions) (cf. FIG. 3).

Objective response rate (ORR) per RECIST 1.1 were observed in 50% [32.9; 67.1] of the patients in the IT arm vs 65.0% [0.41:0.85] in the IV arm.

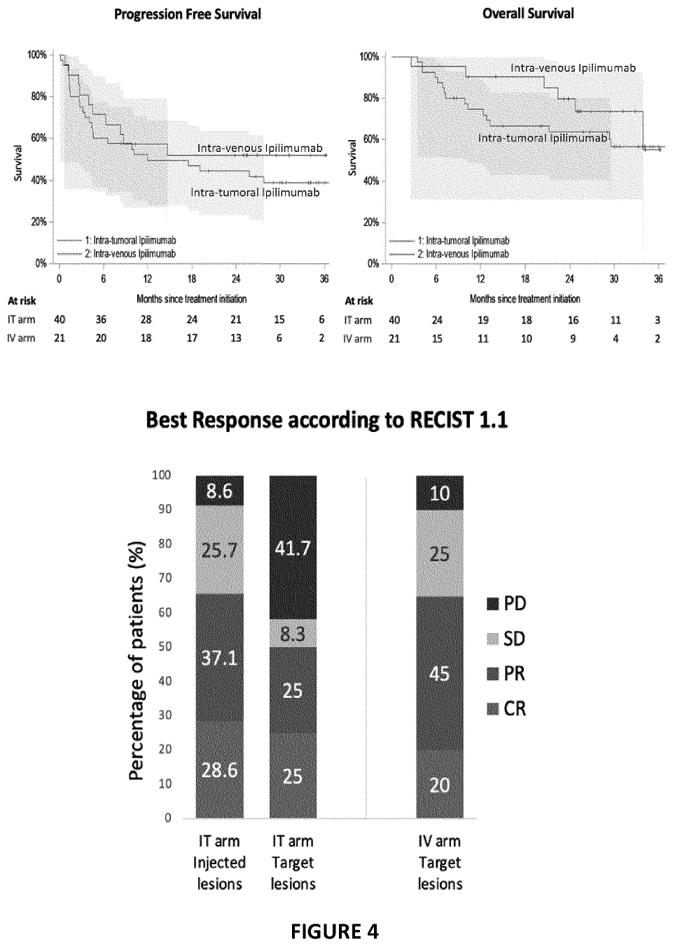

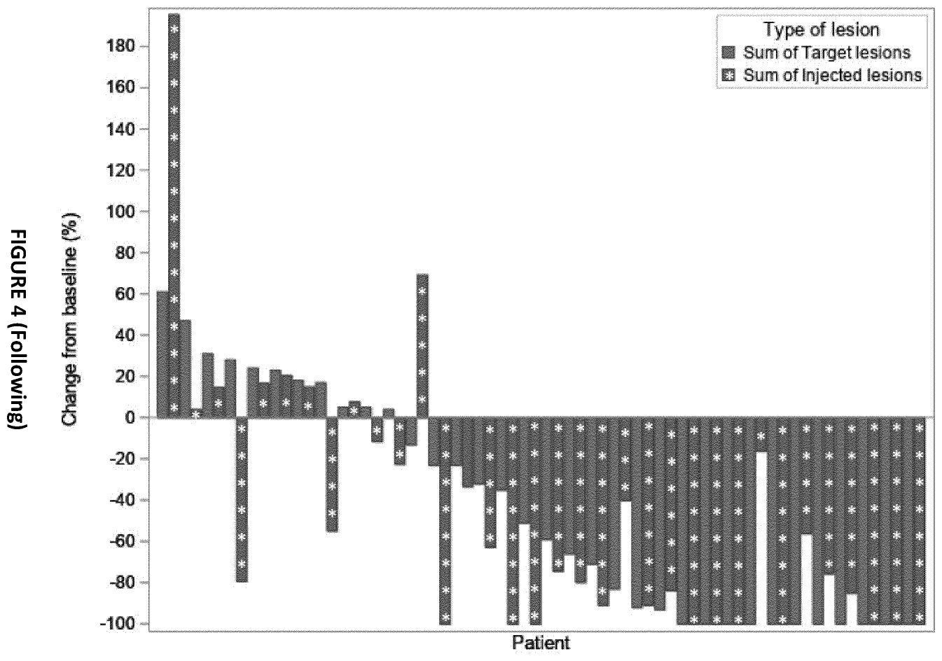

In the IT arm, 65.7% of the injected tumors showed a complete response (CR) or partial response (PR) (cf. FIG. 4).

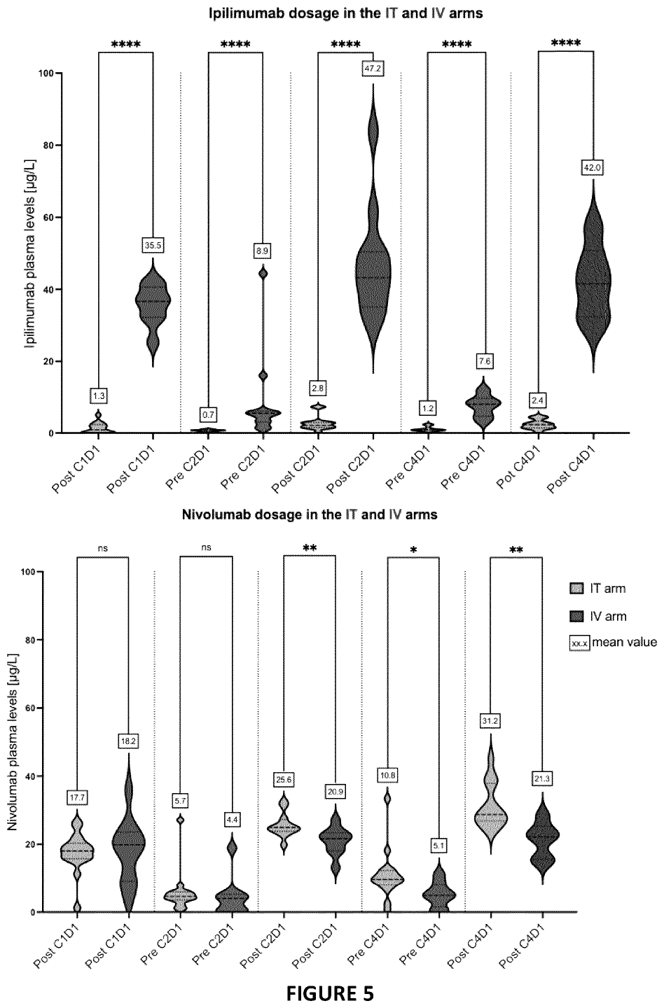

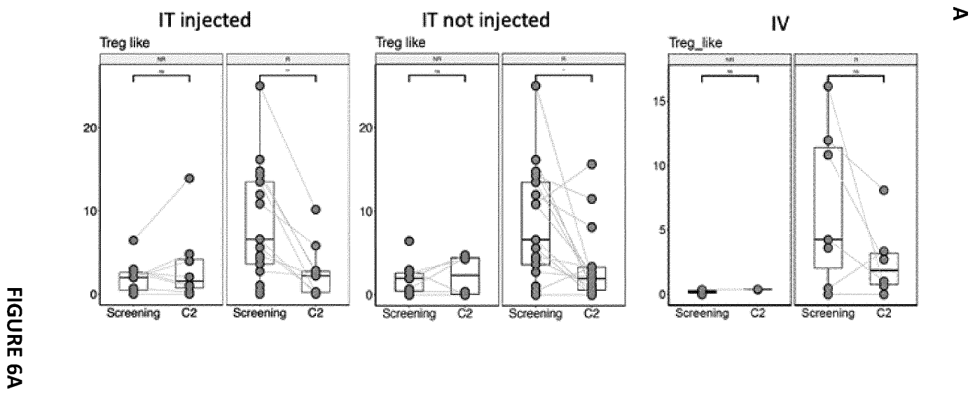

Serum Ipi concentrations were much lower in the IT arm (: 10) (cf. FIG. 5).

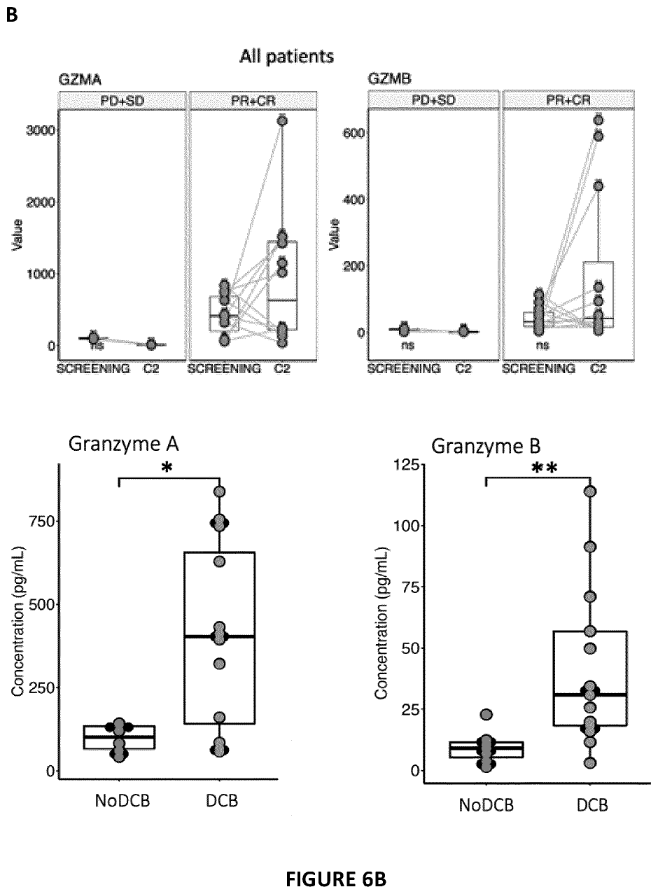

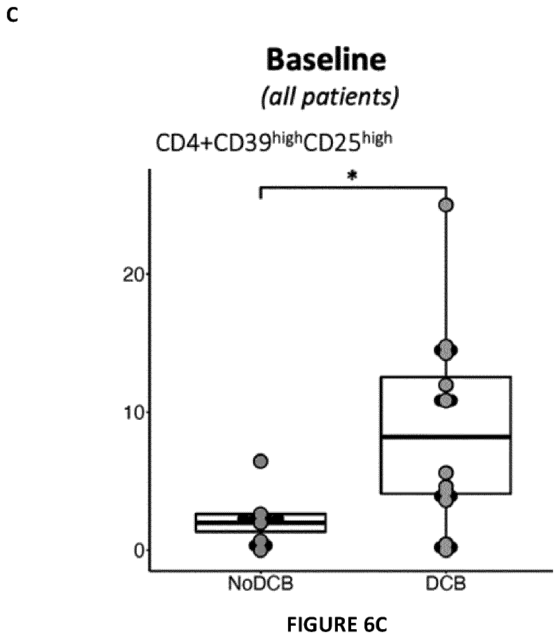

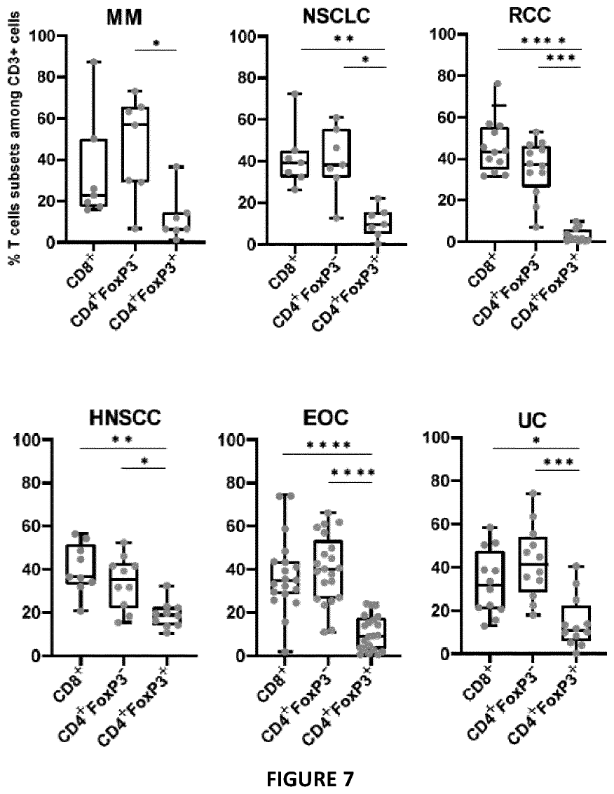

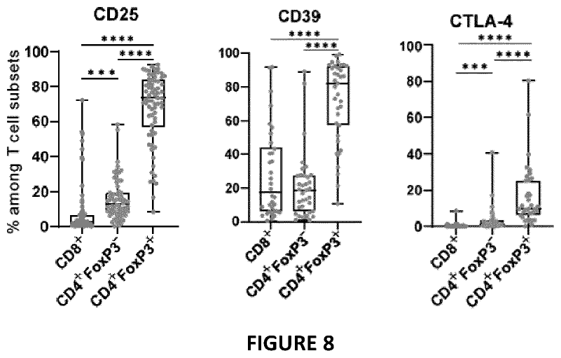

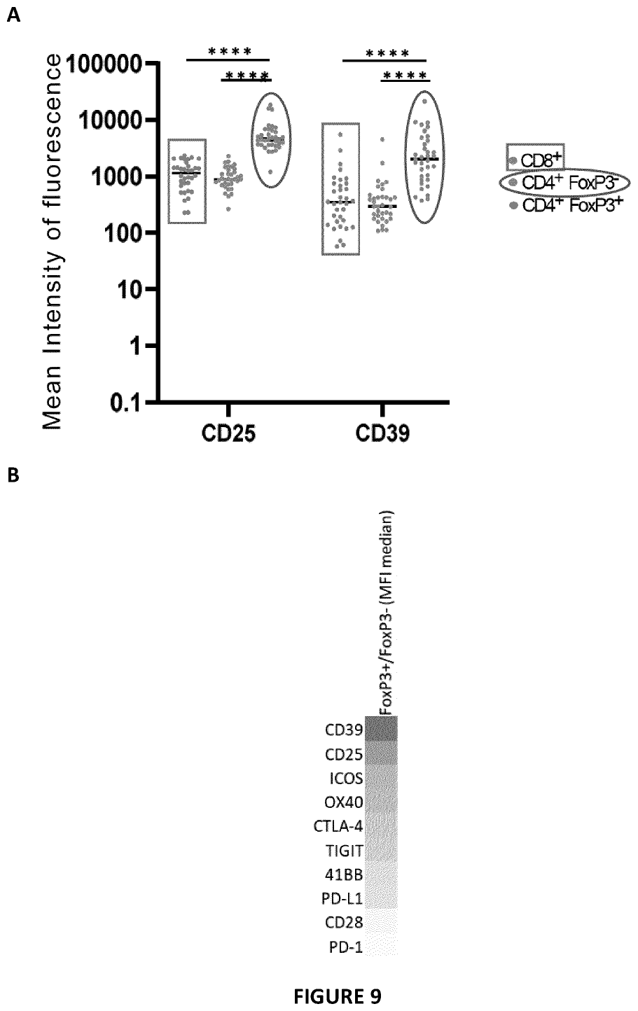

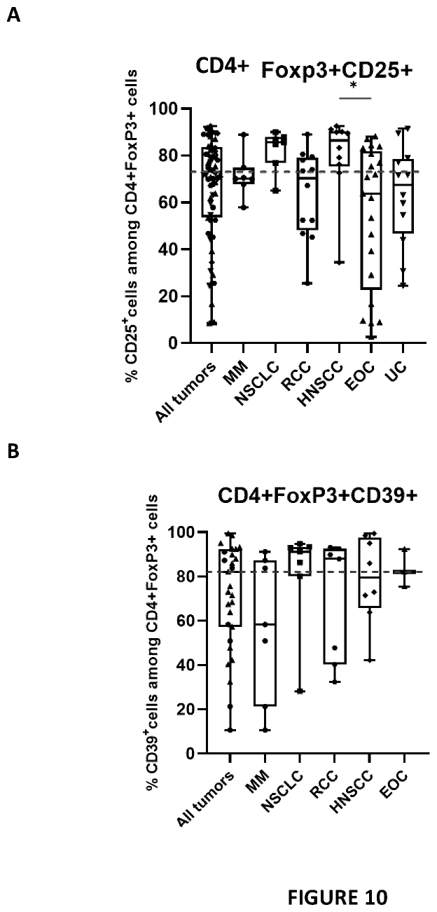

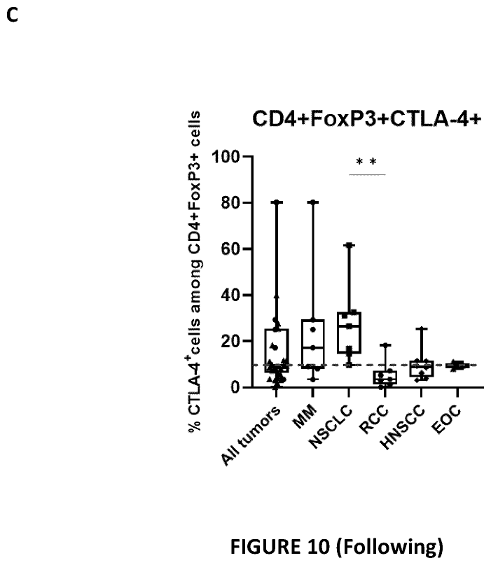

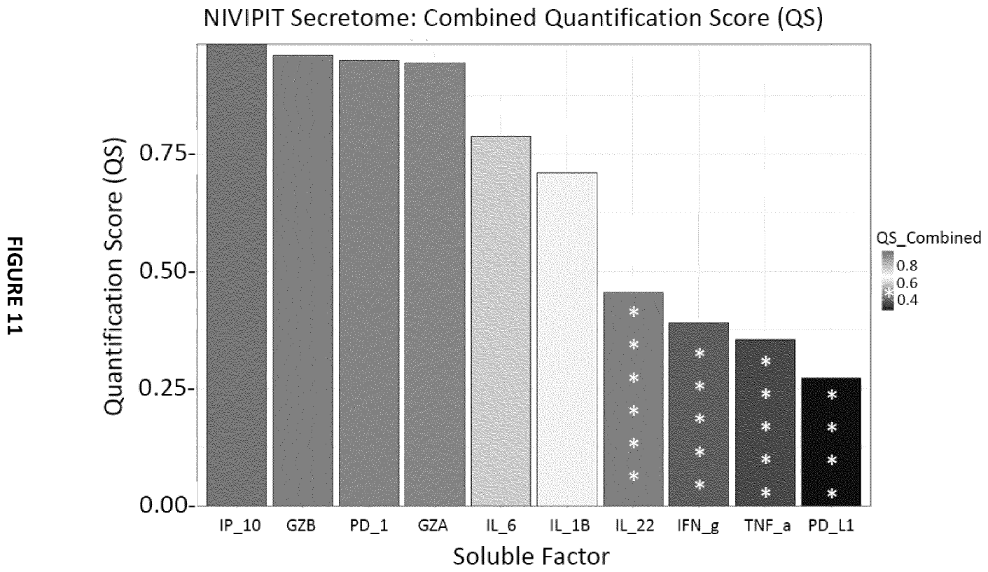

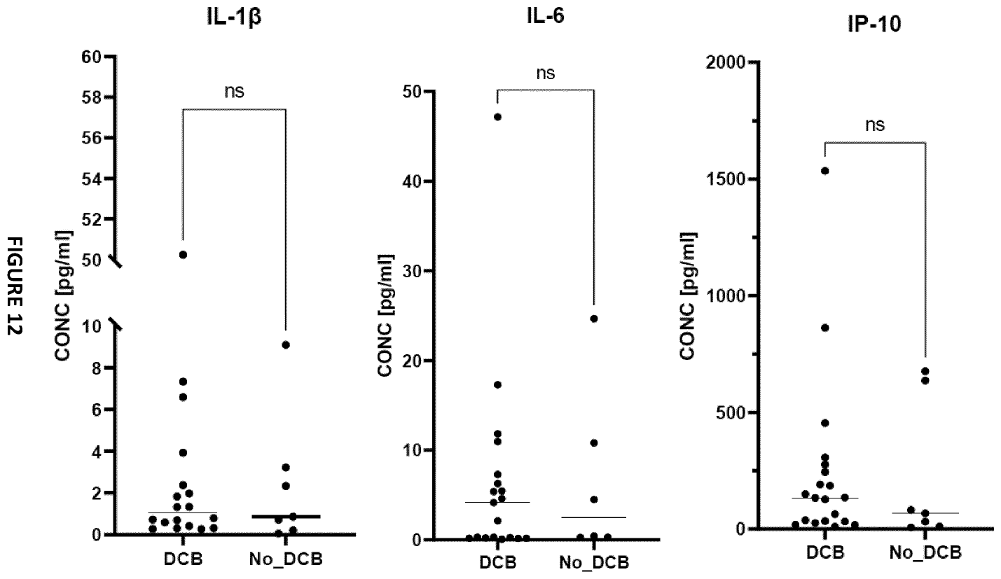

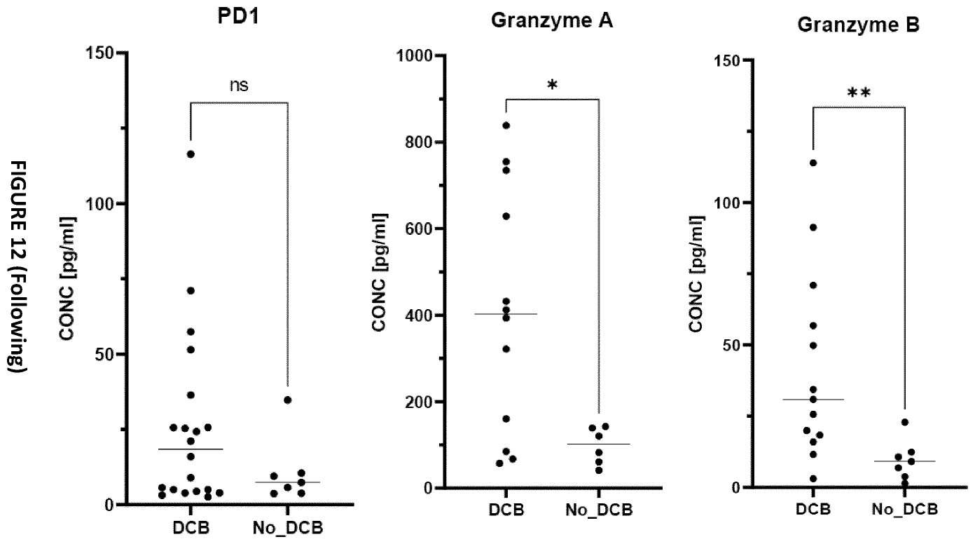

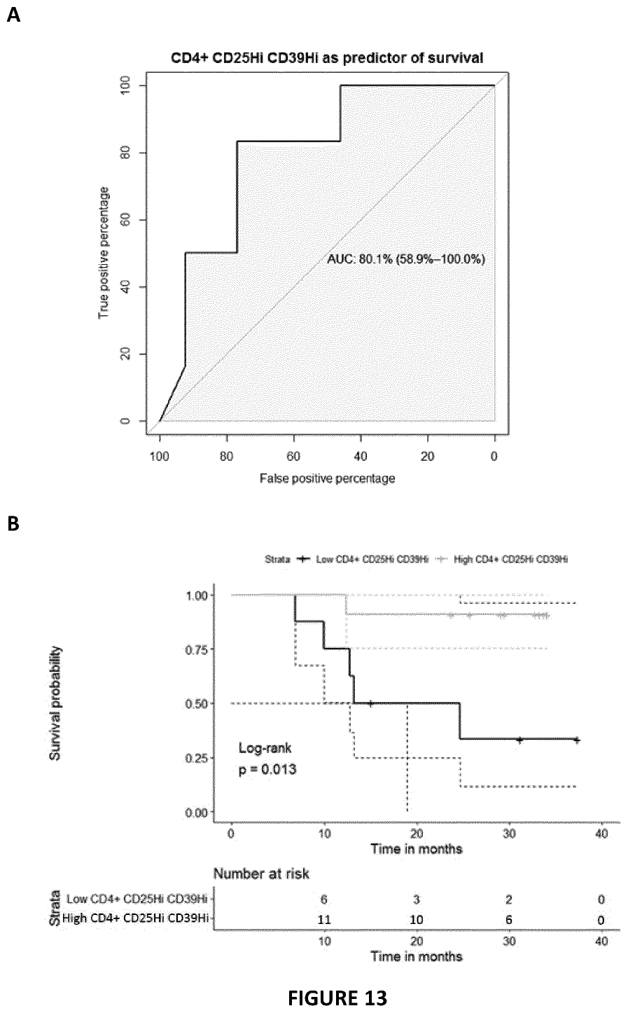

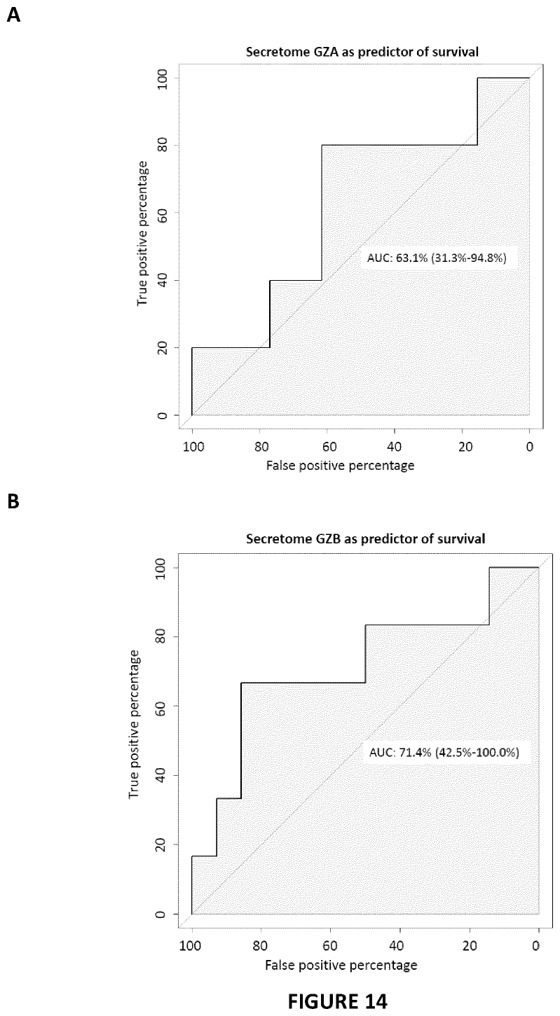

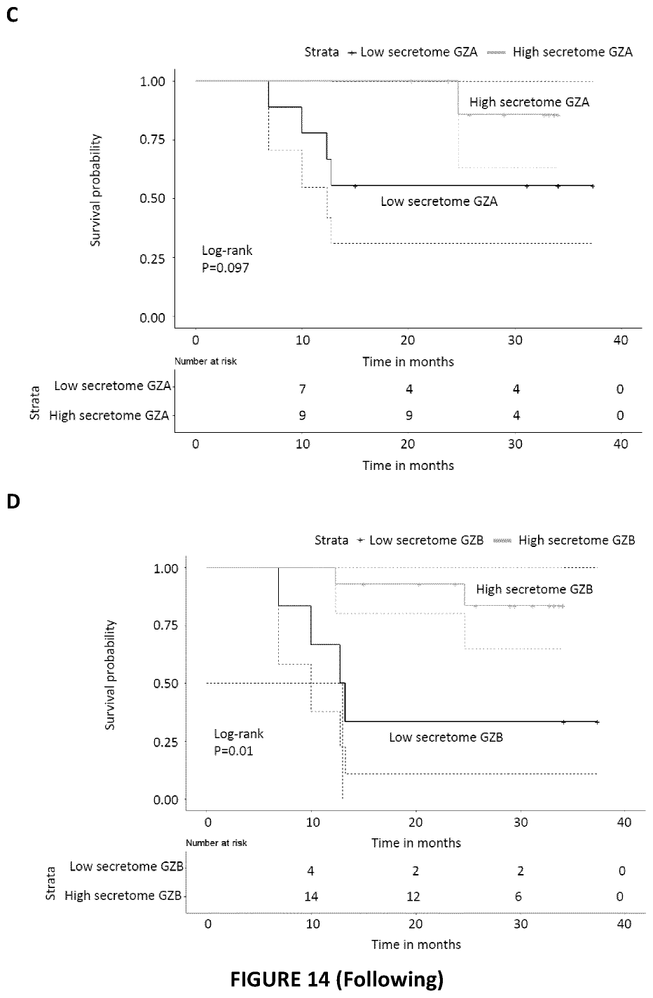

Before beginning a second step of treatment with the same immunotherapeutic treatment (i.e., at “C2”), patients in both arms had significant decreased circulating naïve regulatory T cells (Tregs) independently from tumor responses. Presence of intratumoral CD25hi CD39hi activated Tregs that decreased significantly upon IT injection only in responders (including DCB patients), was predictive of the overall tumor response in the IT arm (cf. FIG. 6A). Moreover, both granzyme A and granzyme B concentrations in tumor secretome at baseline were significantly higher in responders (including DCB patients) than non-responders in both arms (cf. FIG. 6B).

### Conclusions

IT Ipi in combination with IV Nivo is not only safe but could reduce grade 3 (gr3) toxicity of the Immune Checkpoint Blockers (“ICB”) combination. The high response rate in injected lesions was associated with the reduction of intra-tumoral activated Treg and prompts a use of the herein identified new biomarkers (CD4+CD25highCD39high T cells, also herein identified as “activated Tregs” or “CD25hi CD39hi activated Tregs”: as well as Granzyme B) in the oligometastatic and neoadjuvant setting to determine the sensitivity or resistance of a subject to immunotherapy, preferably to an immunotherapy combining at least two immunotherapeutic agents, in particular to anti-PD1 and anti-CD4 combined immunotherapy. In addition, direct assessment of cytolytic and regulatory pathways on fresh biopsies represents a novel, simple and rapid strategy to predict treatment efficacy.

### Example 2-CD4⁺CD25highCD39High T Cells Biomarker Identified in Other Cancers

In order to better define what is the prevalence of the CD4+CD25+CD39+ T-cell population in cancer, inventors prospectively phenotype 35 freshly resected primary tumors.

TABLE 2
Patient Characteristics

| | | MM | | NSCLC | | RCC | | HNSCC | | EOC | | UC | |
|---|---|---|---|---|---|---|---|---|---|---|---|---|---|
| | | n | % | n | % | n | % | n | % | n | % | n | % |
| Total | | 7 | 9% | 9 | 12% | 12 | 16% | 11 | 14% | 22 | 29% | 12 | 16% |
| Gender | Female | 3 | 43% | 4 | 44% | 2 | 17% | 4 | 36% | 22 | 100% | 1 | 8% |
| | Male | 3 | 43% | 4 | 44% | 10 | 83% | 7 | 64% | 0 | 0% | 9 | 75% |
| | Missing | 1 | 14% | 1 | 11% | 0 | 0% | 0 | 0% | 0 | 0% | 2 | 17% |
| Age | Median | 50 | — | 68 | — | 57 | — | 63 | — | 55 | — | 78 | — |
| (years) | Range | 40-63 | — | 63-79 | — | 33-67 | — | 21-89 | — | 19-74 | — | 47-89 | — |
| | Missing | 1 | 14% | 1 | 11% | 4 | 33% | 0 | 0% | 0 | 0% | 2 | 17% |
| Metastatic | N+ | 7 | 100% | 5 | 56% | 1 | 8% | 8 | 73% | 11 | 50% | 5 | 42% |
| Lymph | N− | 0 | 0% | 3 | 33% | 11 | 92% | 2 | 18% | 5 | 23% | 5 | 42% |
| Nodes | Nx | 0 | 0% | 0 | 0% | 0 | 0% | 1 | 9% | 6 | 27% | 0 | 0% |
| | Missing | 0 | 0% | 1 | 11% | 0 | 0% | 0 | 0% | 0 | 0% | 2 | 17% |
| Status at | Primary | 0 | 0% | 8 | 89% | 6 | 50% | 10 | 91% | 22 | 100% | 0 | 0% |
| surgery | Relapsing | 7 | 100% | 0 | 0% | 1 | 8% | 1 | 9% | 0 | 0% | 0 | 0% |
| | Missing | 0 | 0% | 1 | 11% | 5 | 42% | 0 | 0% | 0 | 0% | 12 | 100% |
| NLR | Low (<2.59) | 0 | 0% | 2 | 22% | 3 | 25% | 6 | 55% | 11 | 50% | 0 | 0% |
| | High (≥2.59) | 0 | 0% | 6 | 67% | 5 | 42% | 4 | 36% | 9 | 41% | 0 | 0% |
| | Missing | 7 | 100% | 1 | 11% | 4 | 33% | 1 | 9% | 2 | 9% | 12 | 100% |
| dNLR | Low (<1.65) | 0 | 0% | 2 | 22% | 4 | 33% | 6 | 55% | 17 | 77% | 0 | 0% |
| | High (≥1.65) | 0 | 0% | 6 | 67% | 4 | 33% | 4 | 36% | 3 | 14% | 0 | 0% |
| | Missing | 7 | 100% | 1 | 11% | 4 | 33% | 1 | 9% | 2 | 9% | 12 | 100% |
| Assays | Cyrometry T cells | 7 | 100% | 7 | 78% | 12 | 100% | 10 | 91% | 21 | 95% | 12 | 100% |
| | Cytometry ICPs | 7 | 100% | 6 | 67% | 7 | 58% | 8 | 73% | 4 | 18% | 0 | 0% |
| | scRNAseq | 0 | 0% | 2 | 22% | 0 | 0% | 1 | 9% | 1 | 5% | 0 | 0% |

First, inventors have identified the proportion of CD4+FOXP3+ regulatory T-cells (Tregs) 5 among the CD3+ cells population in freshly resected primary tumors of different histology (FIG. 7) and the proportion of CD25+, CD39+ and CTLA4+ cells within CD8+, CD4+Foxp3− et CD4+Foxp3+ cells population (FIG. 8). The level of expression of CD25 and CD39 have been measured (FIG. 9A). CD39 and CD25 are the highest checkpoint expressed in terms of Ratio of Mean Fluorescence Intensity (MFI) between Foxp3+ and Foxp3− cells (FIG. 9B) 10

Inventors have demonstrated the presence of this new biomarker in different types of cancer. They showed that more than 75% of CD4+Foxp3⁺ T cells express CD25 (FIG. 10A) and more than 80% of CD4+Foxp3⁺ T cells express CD39 (FIG. 10B) in different types of tumor: Malignant Mesothelioma (MM), Non-Small Cell Lung Cancer (NSCLC), Renal Cell Carcinoma (RCC), Head and Neck Squamous Cell Carcinoma (HNSCC), Epithelial Ovarian Cancer (EOC) and Urothelial Carcinoma (UC).

### Example 3—Secretome Cytokines Measurement

To evaluate soluble factors in patient's supernatant of biopsies, a Quickplex SQ120 platform enabling highly sensitive electrochemiluminescent detection (Meso Scale Discovery, Rockville, MD) was used following the manufacturer's instructions. The analytes measured were: Interleukins (IL-1B, IL-2RA, IL-6, IL-22), Interferons (IFN-γ): IFN-γ-inducible protein-10 (IP-10): Tumor Necrosis Factors (TNF-α): Vascular Endothelial Growth Factor (VEGF): Immune checkpoint soluble proteins (PD-1, PD-L1): Cytotoxic Granules (Granzyme A and B). Absolute concentrations of soluble analytes (in pg/mL) in patient samples were calculated by use of a four-point-fit calibration curve of the standard dilutions (MSD DISCOVERY WORKBENCH analysis software) and were considered “detectable” if both runs of each sample had a signal greater than the analyte- and plate-specific lower limit of detection (LLOD) (FIG. 11). Inventors consider that a cytokine/soluble factor is measurable when the Quantification Score (QS) is above 0.5. Best QS were found for IP-10, Granzyme A and B, PD-1, IL-6, IL-1B, IL-2Ra and VEGF in the secretome of biopsies (FIG. 12).

### Example 4: Highly-Specific Predictive Value of CD4+CD25highCD39High Cells, Granzyme A and Granzyme B

An optimal cut-off value was determined using the Youden index, with the objective of maximizing the AUC of the ROC Curve for the CD4+CD25highCD39high at baseline. The optimal cut-off value at baseline is 3.61% CD4+CD25highCD39high with AUC 80.1% (FIG. 13A). The resulting AUC of the ROC curve demonstrates a highly-specific and moderately-sensitive predictive value of CD4+CD25highCD39high frequencies.

Kaplan Meyeur Survival analysis demonstrates that patients with “low” CD4+CD25highiCD39high frequencies at baseline are at increased risk of death compared to those with “high” frequencies (FIG. 13 B).

An optimal cut-off value was determined using the Youden index, with the objective of maximizing the AUC of the ROC Curve for the Granzyme A (FIG. 14A) and Granzyme B (FIG. 14 B) in the secretome at baseline. The optimal Granzyme A cut-off value is 160 pg/ml with AUC 63%, the optimal Granzyme B cut-off value is 11, 6 μg/ml with AUC 71%.

Kaplan Meyeur Survival analysis demonstrate that patients with “low” secretome Granzyme A frequencies (FIG. 14 C) and secretome Granzyme B frequencies (FIG. 14 D) at baseline are at increased risk of death compared to those with “high” frequencies.

### REFERENCES

Ilie, M., Long-Mira, E., Bence, C., Butori, C., Lassalle, S., Bouhlel, L., Fazzalari, L., Zahaf, K., Lalvée, S., Washetine, K., et al. (2016). Comparative study of the PD-L1 status between surgically resected specimens and matched biopsies of NSCLC patients reveal major discordances: a potential issue for anti-PD-L1 therapeutic strategies. Ann. Oncol. 27, 147-153.
Kerr, K. M., Tsao, M.-S., Nicholson, A. G., Yatabe, Y., Wistuba, I. I., and Hirsch, F. R. (2015). Programmed Death-Ligand 1 Immunohistochemistry in Lung Cancer: In what state is this art? J. Thorac. Oncol. 10, 985-989.
Larkin, J., Chiarion-Sileni, V., Gonzalez, R., Grob, J.-J., Rutkowski, P., Lao, C. D., Cowey, C. L., Schadendorf, D., Wagstaff, J., Dummer, R., et al. (2019). Five-Year Survival with Combined Nivolumab and Ipilimumab in Advanced Melanoma. N. Engl. J. Med. 381, 1535-1546
Marabelle, A., Fakih, M., Lopez, J., Shah, M., Shapira-Frommer, R., Nakagawa, K., Chung, H. C., Kindler, H. L., Lopez-Martin, J. A., Miller, W. H. J., et al. (2020). Association of tumour mutational burden with outcomes in patients with advanced solid tumours treated with pembrolizumab: prospective biomarker analysis of the multicohort, open-label, phase 2 KEYNOTE-158 study. Lancet. Oncol. 21, 1353-1365.
Tumeh, P. C., Harview, C. L., Yearley, J. H., Shintaku, I. P., Taylor, E. J. M., Robert, L., Chmielowski, B., Spasic, M., Henry, G., Ciobanu, V., et al. (2014). PD-1 blockade induces responses by inhibiting adaptive immune resistance. Nature 515, 568-571.
Vanhersecke, L., Brunet, M., Guégan, J.-P., Rey, C., Bougouin, A., Cousin, S., Le Moulec, S., Besse, B., Loriot, Y., Larroquette, M., et al. (2021). Mature tertiary lymphoid structures predict immune checkpoint inhibitor efficacy in solid tumors independently of PD-L1 expression. Nat. Cancer 2, 794-802.
Wagle, N., Emery, C., Berger, M. F., Davis, M. J., Sawyer, A., Pochanard, P., Kehoe, S. M., Johannessen, C. M., Macconaill, L. E., Hahn, W. C., et al. (2011). Dissecting therapeutic resistance to RAF inhibition in melanoma by tumor genomic profiling. J. Clin. Oncol. 29, 3085-3096.

1-14. (canceled)

15. An in vitro method of predicting, assessing or monitoring the sensitivity or resistance of a subject having a cancer to an immunotherapy combining at least two immunotherapeutic agents, in particular an anti-PD-1 monoclonal antibody and an anti-CTLA4 antibody, wherein the method comprises a step of determining, before any immunotherapeutic treatment step in the subject, in a tumor sample of the subject, the presence of CD4⁺CD25highCD39high T cells, the presence of CD4⁺CD25highCD39high T cells in the tumor sample being indicative of sensitivity of the subject to the immunotherapy, and the absence of CD4⁺CD25highCD39high T cells in the tumor sample being indicative of resistance of the subject to the immunotherapy.

16. The in vitro method according to claim 15, wherein CD4⁺CD25highCD39high T cell further expresses at least one additional marker selected from CD3, CD45, CD127 and Foxp3.

17. The in vitro method according to claim 16, wherein the CD4⁺CD25highCD39high T cell is a CD127low T cell.

18. The method according to claim 15, wherein the anti-PD-1 monoclonal antibody is selected from nivolumab and pembrolizumab.

19. The method according to claim 15, wherein the anti-CTLA4 monoclonal antibody is selected from ipilimumab and tremelimumab.

20. The method according to claim 15, wherein the cancer is selected from melanoma, lung, head and neck cancer, renal cancer and bladder cancer.

21. The method according to claim 15, wherein the tumor sample is a fresh tumor sample biopsy or a tumor sample biopsy which has not been frozen.

22. The method according to claim 21, wherein the method comprises a step of dosing via ELISA at least one marker selected from VEGFA, IL6, CXCL8, granzyme, in the supernatant of the fresh tumor sample biopsy after an incubation step of at least one minute.

23. An in vitro method of predicting, assessing or monitoring the sensitivity or resistance of a subject having a cancer to an immunotherapy combining at least two immunotherapeutic agents, in particular an anti-PD-1 monoclonal antibody and an anti-CTLA4 antibody, after one or several treatment steps with the immunotherapy in the subject, wherein the method comprises a step a) of determining, in a tumor sample of the subject, the expression level of CD4⁺CD25highCD39high T cells, and a step b) of comparing said CD4⁺CD25highCD39high T cells level to a CD4⁺CD25highCD39high T cells reference expression level, an expression level of CD4⁺CD25highCD39high T cells below the CD4⁺CD25highCD39high T cells reference expression level being indicative of sensitivity of the subject to the immunotherapy and an expression level of CD4⁺CD25highCD39high T cells above the CD4⁺CD25highCD39high T cells reference expression level being indicative of resistance of the subject to the immunotherapy.

24. The in vitro method according to claim 23, wherein the CD4⁺CD25highCD39high T cells reference expression level is the level of CD4⁺CD25highCD39high T cells in the tumor of the subject before any immunotherapeutic treatment step in the subject.

25. The in vitro method according to claim 23, wherein CD4⁺CD25highCD39high T cell further expresses at least one additional marker selected from CD3, CD45, CD127 and Foxp3.

26. The in vitro method according to claim 25, wherein the CD4⁺CD25highCD39high T cell is a CD127low T cell.

27. The method according to claim 23, wherein the anti-PD-1 monoclonal antibody is selected from nivolumab and pembrolizumab.

28. The method according to claim 23, wherein the anti-CTLA4 monoclonal antibody is selected from ipilimumab and tremelimumab.

29. The method according to claim 23, wherein the cancer is selected from melanoma, lung, head and neck cancer, renal cancer and bladder cancer.

30. The method according to claim 23, wherein the tumor sample is a fresh tumor sample biopsy or a tumor sample biopsy which has not been frozen.

31. The method according to claim 30, wherein the method comprises a step of dosing via ELISA at least one marker selected from VEGFA, IL6, CXCL8, granzyme, in the supernatant of the fresh tumor sample biopsy after an incubation step of at least one minute.

32. The method according to claim 30, wherein the fresh tumor sample biopsy or tumor sample biopsy which has not been frozen, has been dissociated with both enzymatic and mechanical procedures before being stained and used.

33. A method of selecting an appropriate therapeutic treatment for a subject having a cancer, which method comprises a step of predicting or assessing the sensitivity of a subject having a cancer to an immunotherapy combining at least two immunotherapeutic agents using a method according to claim 15.

34. A method of selecting or disqualifying a subject having a cancer for inclusion in a clinical trial, the clinical trial being for evaluating an immunotherapy combining at least two immunotherapeutic agents, which method comprises a step of predicting or assessing the sensitivity of a subject having a cancer to an immunotherapy combining at least two immunotherapeutic agents using a method according to claim 15.
